(19) United States
(12) Patent Application Publication
(10) Pub. No.: US 2008/0268041 A1
(43) Pub. Date: Oct. 30, 2008

(54) COMBINATION OF PROGESTERONE-RECEPTOR ANTAGONIST TOGETHER WITH NONE-STEROIDAL ANTIESTROGEN FOR USE IN BRCA MEDIATED DISEASES

(76) Inventors: Jens Hoffmann, Muhlenbeck (DE); Daniel Korr, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/105,357

(22) Filed: Apr. 18, 2008

Related U.S. Application Data
(60) Provisional application No. 60/914,385, filed on Apr. 27, 2007.

(30) Foreign Application Priority Data
Apr. 23, 2007 (EP) 07090082.4

(51) Int. Cl. A61K 9/48; A61K 31/575; A61P 35/00

(52) U.S. Cl. 424/456; 514/171

(57) ABSTRACT

The present invention relates to the combination of the progesterone-receptor antagonist 11β-(4-acetylphenyl)-17β-hydroxy-17α-(1,1,2,2,2-pentafluoroethyl)-estra-4,9-dien-3-one or a pharmaceutically acceptable derivative or analogue thereof, together with at least one none-steroidal antiestrogen and to the use of said combination for the prophylaxis and treatment of BRCA1- or BRCA2-mediated diseases. None-steroidal antiestrogens which can be combined together with the progesterone-receptor antagonist 11β-(4-acetylphenyl)-17β-hydroxy-17α-(1,1,2,2,2-pentafluoroethyl)-estra-4,9-dien-3-one are for example is tamoxifen, raloxifene, droloxifen, toremifen, lasofoxifen, arzoxifen, GW5638, EM-800, idoxifen and basedoxifene.

This application claims the benefit of the filing date of European Application Serial No. 07090082.4 filed Apr. 23, 2007 and U.S. Provisional Application Ser. No. 60/914,385 filed Apr. 27, 2007.

The present invention relates to the combination of the progesterone-receptor antagonist 11β-(4-acetylphenyl)-17β-hydroxy-17α-(1,1,2,2,2-pentafluoroethyl)-estra-4,9-dien-3-one or a pharmaceutically acceptable derivative or analogue thereof, together with at least one pure none-steroidal antiestrogen and to the use of said combination for the prophylaxis and treatment of BRCA1- or BRCA2-mediated diseases.

The progesterone-receptor antagonist 11β-(4-acetylphenyl)-17β-hydroxy-17α-(1,1,2,2,2-pentafluoroethyl)-estra-4,9-dien-3-one, also known as ZK230211 or ZK-PRA,

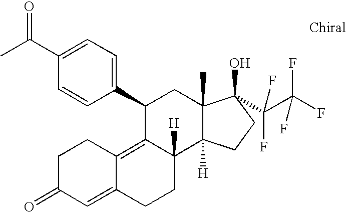

has high antiprogestagenic activity with little or no other endocrinological effects (Fuhrmann, U. et al., J. Med. Chem. 2000, 43, 5010-5016).

BRCA1 and BRCA2 are so-called tumuppressors, genes that in their normal form protect against cancer. One way they do this is by helping cells repair DNA damage that might otherwise result in cancer-causing mutations. In Poole et al., Science, Vol. 314, 12/2006 it is described that the tumuppressor gene BRCA-1- or BRCA2 participates in the degradation of the progesterone receptor, the gene's protein product apparently controls the progesterone growth-promoting action on breast tissue.

It is shown that mifepristone, an unspecific antiprogestin, blocks the development of mammary tumors in mice that have had the rodent version of BRCA1- or BRCA2 inactivated in their mammary glands. It is further postulated that mifepristone mediated inhibition of mammary tumorgenesis in their Brca1/p53-deficient model provides a molecular framework for future clinical evaluation of antiprogesterones as a potential chemopreventive strategy in women who carry BCRA1- or BRCA2 mutations. However, nothing is described with respect to the activity and reaction of 11β-(4-acetylphenyl)-17β-hydroxy-17α-(1,1,2,2,2-pentafluoroethyl)-estra-4,9-dien-3-one in combination with an pure non-steroidal antiestrogen.

Rosen et al. describe that normal BRCA1- or BRCA2 inhibits the action of the progesterone receptor, however nothing is mentioned about the mechanism.

Endocrine therapy represents a mainstay of effective, minimally toxic, palliative treatment for metastatic breast cancer. As a standard palliative treatment of non-operable mammary carcinomas as well as for adjuvant therapy after primary treatment of mammary carcinomas, antiestrogens, such as the non-steroidal antiestrogen tamoxifen, are used. However, tamoxifen cannot cure breast cancer. Thus, fecondary therapy progestins are commonly used. In premenopausal women ovariectomy, tamoxifen and LHRH (luteinizing hormone releasing hormones) analogs achieve comparable results (H. T. Mouridson et al., Eur. J. Cancer Clin. Oncol., 24, pp. 99-105, 1988). Although tamoxifen is widely used for adjuvant therapy of breast cancer, its use as a chemopreventive agent is problematic, because it has been shown that the treatment results in an increase in the incidence of endometrial cancers (I. N. White, Carcinogenesis, 20(7):1153-60, 1999; L. Bergman et al., The Lancet, Vol. 356, Sep. 9, 2000).

Selective Progesterone-receptor antagonists (also termed as antiprogestins) represent a relatively new and promising class of therapeutic agents that could have significant impact on cancer treatment. Certain progesterone-receptor antagonists have recently gained importance in the endocrine therapy of those cancers possessing receptors for progesterone (Nathalie Chabbert-Buffet et al, Human Reproduction Update, Vol. 11, No. 3, 293-307, 2005).

This new strategy in endocrine therapy is based on the antitumor activity of progesterone-receptor antagonists in progesterone receptor-positive human breast cancer cell lines in vitro and in several hormone-dependent mammary tumors of the mouse and rat in vivo. In particular, the antitumor mechanism of the progesterone-receptor antagonists onapristone and mifepristone (RU 486) was investigated using the hormone-dependent MXT mammary tumor model of the mouse as well as the DMBA- and the MNU-induced mammary tumor models of the rat (M. R. Schneider et al., Eur. J. Cancer Clin. Oncol., Vol. 25, No. 4, pp. 691-701, 1989; H. Michna et al., Breast Cancer Research and Treatment 14:275-288, 1989; H. Michna, J. Steroid. Biochem. Vol. 34, Nos 1-6, pp. 447-453, 1989). However, due to low activity and adverse side effects involved with e.g. mifepristone these compounds could not be recommended as a single agent in the management of breast cancer (D. Perrault et al., J. Clin. Oncol. 1996 Oct., 14(10), pp. 2709-2712).

RU 486 is causing severe side effects because of its strong anti-glucocorticoidially activity. This prohibits long term use.

When using RU 486, a further problem is for instance the poor bioavailability when administered orally. Thus, the compound generally had to be administered in high doses, giving rise to possible unfavorable side effects. Moreover, oral administration is desirable with respect to patient convenience and compliance.

Furthermore, there is still a need for combinations that are active not only in the treatment, but also in the prophylaxis of breast cancer and other hormone-dependent diseases.

It has been found that the growth of hormone-dependent tumors depend, among others, e.g. on estrogens, progesterones and even testosterones. For example, most mammary carcinomas exhibit estrogen as well as progesterone receptors. Thus, a combination of progesterone-receptor antagonists together with antiestrogens may be effective in the therapy of pre- and postmenopausal mammary carcinomas.

Combinations with tamoxifen and the antiprogestine have not been disclosed. This might be the result with respect to the weak activity of the antiestrogen part of the combination and from the partial estrogen agonism of certain antiestrogens (like, e.g., tamoxifen).

We have however surprisingly seen a synergistic effects of the inventive combinations. One further advantage is the inhibition of proliferative effects of tamoxifen on the uterus by the combination with the progesterone antagonist. The combination of an anastrazole with tamoxifen has been profen to be less effective than the monotherapy with one of these compound (Ref. ATAC Trial results 2005). Our findings provide evidence that combination of none-steroidal antiprogestines, such as tamoxifen may have in contrast synergistic effects on tumor growth inhibition and survival.

It is thus the object of the present invention to provide a highly efficient tool for prophylaxis and treatment of especially breast cancer development and other diseases dependent upon progesterone in BRCA1- or BRCA2 mutations bearing women, such as ovarian cancer, endometrial cancer, colorectal cancer, gastric cancer, endometriosis, myeloma, myoma and meningioma.

It has now surprisingly been found that 11β-(4-acetylphenyl)-17β-hydroxy-17α-(1,1,2,2,2-pentafluoroethyl)-estra-4,9-dien-3-one in combination together with at least one pure none-steroidal antiestrogen can be used for the prophylaxis and treatment of BRCA1- or BRCA2-mediated breast cancer, ovarian cancer, endometrial cancer, colorectal cancer, gastric cancer, endometriosis, myeloma, myoma and meningioma.

It has now further most surprisingly been found that the combination of the progesterone-receptor antagonist 11β-(4-acetylphenyl)-17β-hydroxy-17α-(1,1,2,2,2-pentafluoroethyl)-estra-4,9-dien-3-one together with a none-steroidal antiestrogen shows a synergistic effect when compared to the inhibition of the progesterone-receptor antagonist, or the pure antiestrogens alone.

Antiestrogenes which can be combined together with the compound 11β-(4-acetylphenyl)-17β-hydroxy-17α-(1,1,2,2,2-pentafluoroethyl)-estra-4,9-dien-3-one are for example tamoxifen, raloxifene, droloxifen, toremifen, lasofoxifen, arzoxifen, GW5638*), EM-800**), idoxifen and basedoxifene.

*) chemical structure disclosed in Wilson et al., Endocrinology 138, 3901, (1997) and Wu et al., Mol. Cell, 18, 413, (2005)

**) chemical structure disclosed in Labrie et al., J. Steroid Biochem. Mol. Biol. 79, 213, (2001)

It has further been found that 11β-(4-acetylphenyl)-17β-hydroxy-17α-(1,1,2,2,2-pentafluoroethyl)-estra-4,9-dien-3-one in combination with a none-steroidal antiestrogen were accompanied by increased apoptosis of tumor cells, a particularly advantageous mechanism of action for the prevention or treatment of mammary carcinoma and other hormone-dependent diseases, where an indicator of high risk is an increased amount of tumor cells in the S-phase of the cell cycle. Such other hormone-dependent diseases may include ovarian cancer, endometrial cancer, myeloma, lung cancer, meningioma, i.e., diseases which substantially originate or are influenced by the presence of hormone receptors and/or hormone-dependent pathways.

Further, the addition of the progesterone-receptor antagonist prevent proliferative effects of tamoxifen on the uterus.

The invention furthermore relates to the use of the combination for the preparation of a medicament for prophylaxis and treatment of cancer in BRCA1 and BRCA2 mutation bearing women, as well as for the treatment of other hormone-dependent conditions. In particular the combination of 11β-(4-acetylphenyl)-17β-hydroxy-17α-(1,1,2,2,2-pentafluoroethyl)-estra-4,9-dien-3-one together with a pure none-steroidal antiestrogen has been shown to effectively inhibit the growth of such tumors as compared to the progesterone-receptor antagonist, or pure antiestrogen alone.

In another aspect, the present invention provides a method for prophylaxis and treatment of breast cancer and other hormone-dependent diseases in a mammal, in particular a human, in need of such treatment because of mutations in the BRCA1 or BRCA2 gene, said method comprising administering a pharmaceutically effective amount of a composition comprising the progesterone-receptor antagonist 11β-(4-acetylphenyl)-17β-hydroxy-17α-(1,1,2,2,2-pentafluoroethyl)-estra-4,9-dien-3-one or a pharmaceutically acceptable derivative or analogue thereof, and at least one pure none-steroidal antiestrogen, or to a mammal in need thereof. 11β-(4-acetylphenyl)-17β-hydroxy-17α-(1,1,2,2,2-pentafluoroethyl)-estra-4,9-dien-3-one or a pharmaceutically acceptable derivative or analogue thereof can be used according to the present invention in combination with at least one pure none-steroidal antiestrogen.

Although progesterone-receptor antagonist 11β-(4-acetylphenyl)-17β-hydroxy-17α-(1,1,2,2,2-pentafluoroethyl)-estra-4,9-dien-3-one is the preferred progesterone-receptor antagonist for purposes of the present invention, this does not exclude the possibility to use other suitable progesterone-receptor antagonists as well.

With regard to the superiority of the inventive combination over the prior art, it is especially favorable that the progesterone-receptor antagonist 11β-(4-acetylphenyl)-17β-hydroxy-17α-(1,1,2,2,2-pentafluoroethyl)-estra-4,9-dien-3-one shows only very weak or no endocrine side effects, such as e.g. androgen, estrogen or antiglucocorticoid activity.

Due to the high bioavailability of the combination according to the present invention comprising the progesterone-receptor antagonist 11β-(4-acetylphenyl)-17β-hydroxy-17α-(1,1,2,2,2-pentafluoroethyl)-estra-4,9-dien-3-one and the pure antiestrogens, including their pharmaceutically acceptable derivatives or analogues thereof, it is possible that the combination can be administered orally.

The oral administration has the advantage of improved convenience and patient compliance. As a further favorable consequence, the combination of the present invention is well tolerated. Partial agonism is commonly associated with undesirable side effects, such as for example in the case of the partial antiestrogen tamoxifen an increase in the incidence of endometrial cancers (see I. N. White, Carcinogenesis, 20(7):1153-60, 1999; L. Bergman et al., The Lancet, Vol. 356, Sep. 9, 2000, 881-887) as well as the antiglucocorticoid effects and certain toxic side effects related to the administration of the prior art progesterone-receptor antagonist mifepristone (see D. Perrault et al., J. Clin. Oncol. 1996 Oct., 14(10), pp. 2709-2712; L. M. Kettel et al., Fertil. Steril. 1991 September, 56(3), pp. 402-407; X. Bertagna, Psychoneuroendocrinology 1997, 22 Suppl. 1; pp. 51-55).

The pure antiestrogens if used in the amounts according to the present invention will not show the undesired side effects associated with the partial antiestrogens.

Optionally, the progesterone-receptor antagonist 11β-(4-acetylphenyl)-17β-hydroxy-17α-(1,1,2,2,2-pentafluoroethyl)-estra-4,9-dien-3-one and the pure none-steroidal antiestrogen can additionally be combined with further pharmacologically active agents, such as cytotoxic agents.

The manufacture of the medicaments/pharmaceutical compositions may be performed according to methods known in the art. Commonly known and used adjuvants, as well as further suitable carriers or diluents may be used.

Suitable carriers and adjuvants may be such as recommended for pharmacy, cosmetics and related fields in: Ullmann's Encyclopedia of Technical Chemistry, Vol. 4, (1953), pp. 1-39; Journal of Pharmaceutical Sciences, Vol. 52 (1963), p. 918ff; H. v. Czetsch-Lindenwald, “Hilfsstoffe für Pharmazie und angrenzende Gebiete”; Pharm. Ind. 2, 1961, p. 72ff; Dr. H. P. Fiedler, Lexikon der Hilfsstoffe für Pharmazie, Kosmetik und angrenzende Gebiete, Cantor K G, Aulendorf in Württemberg, 1971.

The inventive combination also comprises pharmaceutical compositions, which can be prepared by known methods of preparing galenics for oral, parenteral, e.g. intraperitoneal, intramuscular, subcutaneous or percutaneous application. The inventive combination can also be implanted into tissue.

The inventive combination can also be administered in the form of tablets, pills, dragees, gel capsules, granules, suppositories, implants, injectable sterile aqueous or oily solutions, suspensions or emulsions, ointments, creams, gels, patches for transdermal administration, formulations suitable for administration by inhalation, for instance nasal sprays or by intravaginal (e.g. vaginal rings) or intrauterine systems (diaphragms, loops).

For the preparation of the pharmaceutical compositions for oral administration, the active agents suitable for the purposes of the present invention as defined above can be admixed with commonly known and used adjuvants and carriers such as for example, gum arabic, talcum, starch, sugars like e.g. mannitose, methyl cellulose, lactose, gelatin, surface-active agents, magnesium stearate, aqueous or non-aqueous excipients, paraffin derivatives, crosslinking agents, dispersants, emulsifiers, lubricants, conserving agents and flavoring agents (e.g., ethereal oils). In the pharmaceutical composition, the progesterone-receptor antagonist and the pure antiestrogen may be dispersed in a microparticle, e.g. a nanoparticulate, composition.

In order to further enhance the bioavailability of the active agents, the active agents suitable for the purposes of the present invention as defined above can also be formulated as cyclodextrin clathrates by reacting them with α-, β- or γ-cyclodextrines or derivatives thereof according to the method as disclosed in PCT/EP95/02656.

For parenteral administration the active agents suitable for the purposes of the present invention as defined above can be dissolved upended in a physiologically acceptable diluent, such as e.g., oils with or without solubilizers, surface-active agents, dispersants or emulsifiers. As oils for example and without limitation, olive oil, peanut oil, cottonseed oil, soybean oil, castor oil and sesame oil may be used.

The pharmaceutical compositions according to the present invention can also be administered via a depot injection or an implant preparation, optionally sustained delivery of the active agent(s).

Implants can comprise as inert materials e.g. biologically degradable polymers synthetic silicones such as e.g. silicone rubber.

For percutaneous applications, the active agent(s) may also be formulated into adhesives.

The preferred mode of administration is oral administration. The combination according to the present invention are particularly suitable for oral administration.

The inventive combination can be administered by applying the progesterone-receptor antagonist 11β-(4-acetylphenyl)-17β-hydroxy-17α-(1,1,2,2,2-pentafluoroethyl)-estra-4,9-dien-3-one together with the none-steroidal antiestrogens, or applying the progesterone-receptor antagonist 11β-(4-acetylphenyl)-17β-hydroxy-17α-(1,1,2,2,2-pentafluoroethyl)-estra-4,9-dien-3-one separately from the none-steroidal antiestrogens, for example the progesterone-receptor 11β-(4-acetylphenyl)-17β-hydroxy-17α-(1,1,2,2,2-pentafluoroethyl)-estra-4,9-dien-3-one can be administered subcutaneously or i.m. and the none-steroidal antiestrogens, can be administered orally or vice versa.

The amounts (a “pharmaceutically effective amount”) of the combined active agents to be administered vary within a broad range and depend on the condition to be treated and the mode of administration. They can cover any amount efficient for the intended treatment. Determining a “pharmaceutically effective amount” of the combined active agent is within the purview of a person skilled in the art.

The weight ratio of the progesterone-receptor antagonist 11β-(4-acetylphenyl)-17β-hydroxy-17α-(1,1,2,2,2-pentafluoroethyl)-estra-4,9-dien-3-one to the pure none-steroidal antiestrogen(s), as defined above, can vary within a broad range. They can either be present in equal amounts or one component can be present in excess of the other components. Preferably, 0.1 to 200 mg of the pure none-steroidal antiestrogen or and 0.1 to 100 mg of the progesterone-receptor antagonist 11β-(4-acetylphenyl)-17β-hydroxy-17α-(1,1,2,2,2-pentafluoroethyl)-estra-4,9-dien-3-one are administered in a unit dose, more preferably in a unit dose of 10 to 150 mg of each of the pure none-steroidal antiestrogen or and progesterone-receptor antagonist 11β-(4-acetylphenyl)-17β-hydroxy-17α-(1,1,2,2,2-pentafluoroethyl)-estra-4,9-dien-3-one. In special cases up to 200 mg of the progesterone-receptor antagonist 11β-(4-acetylphenyl)-17β-hydroxy-17α-(1,1,2,2,2-pentafluoroethyl)-estra-4,9-dien-3-one may be administered. The pure none-steroidal antiestrogen and progesterone-receptor antagonist 11β-(4-acetylphenyl)-17β-hydroxy-17α-(1,1,2,2,2-pentafluoroethyl)-estra-4,9-dien-3-one are preferably present in ratios from 100:1 to 1:100. More preferably, they are present in ratios from 4:1 to 1:4.

The progesterone-receptor antagonist 11β-(4-acetylphenyl)-17β-hydroxy-17α-(1,1,2,2,2-pentafluoroethyl)-estra-4,9-dien-3-one and the none-steroidal antiestrogen(s) can be administered either together or separately, at the same time and/sequentially. Preferably they are administered combined in one unit dose. In case they are administered sequentially, preferably the progesterone-receptor antagonist 11β-(4-acetylphenyl)-17β-hydroxy-17α-(1,1,2,2,2-pentafluoroethyl)-estra-4,9-dien-3-one is administered before the pure none-steroidal antiestrogen(s), as defined above.

The combination of the progesterone-receptor antagonist 11β-(4-acetylphenyl)-17β-hydroxy-17α-(1,1,2,2,2-pentafluoroethyl)-estra-4,9-dien-3-one and a pure none-steroidal antiestrogen, or pharmaceutically acceptable derivatives or analogues of these components exerts very strong tumor-inhibiting effects in a panel of hormone-dependent breast cancer models (cf. Example 1). The inhibition is synergistic when compared to the inhibition achieved by these compounds alone.

Medicaments, such as the combination in the various aspects of the invention, that induce apoptosis in cells, for example, in the case of tumor cells, by blocking progression in the G₀G₁-phase, have potential applications for treating and preventing numerous conditions. For example, the combination of progesterone-receptor antagonist 11β-(4-acetylphenyl)-17β-hydroxy-17α-(1,1,2,2,2-pentafluoroethyl)-estra-4,9-dien-3-one and pure none-steroidal antiestrogen(s), may be used for treating those cancers where an indicator of high risk is an increased amount of tumor cells in the S-phase of the cell cycle, such as in breast cancer (see G. M. Clark et al., N. Engl. J. Med. 320, 1989, March, pp. 627-633; L. G. Dressler et al., Cancer 61(3), 1988, pp. 420-427 and literature cited therein).

Without limitation to any theory, the results provided in the example indicate that the main mechanism of the antitumor action of a combination of the progesterone-receptor antagonist 11β-(4-acetylphenyl)-17β-hydroxy-17α-(1,1,2,2,2-pentafluoroethyl)-estra-4,9-dien-3-one and pure none-steroidal antiestrogens, according to the present invention in the tested model is a direct estrogen-receptor and/or progesterone-receptor-mediated antiproliferative effect at the level of the tumor cells, via the induction of terminal differentiation associated with terminal cell death. In this manner, the combination according to the invention appears to be capable of eliminating the intrinsic block in terminal differentiation inherent in malignant tumor cells in progesterone receptor-positive and estrogen-receptor positive tumors.

Using cell cultures it was revealed that the progesterone receptor is degraded less when BRCA1- or BRCA2 activity is knocked down. As a result, the transcriptional activity of progesterone receptor by progesterone is longer and also stronger.

We showed that we could reduce the accelerated PR signaling in BRCA1- or BRCA2 knocked down cells by prophylactic treatment with the instant compounds and combinations. This results in an reduced proliferation of these breast cells.

The loss in control of PR transcription may be one explanation why tumors occur specifically in the breast, ovaries and endometrium meningio organs that specifically dependent on PR, even though the BRCA1- or BRCA2 gene is mutated in cells throughout the body.

Mammary tissue of female mice bearing a similar to human BRCA1- or BRCA2 mutation (and in which p53 gene has been knocked out, showed increased cell proliferation and progesterone receptors expression and develop mammary cancers. Mice treated with the instant compounds, respectively combinations, however, were tumor free.

The effects of the instant compounds, respectively combinations may not only be restricted to tumor tissue but rather to tissue adjacent to <human> breast tumors with BRCA1- or BRCA2 mutations which also shows elevated progesterone expression compared to tissue from normal breast.

The invention is further illustrated in the Examples. The following Examples are, however, not to be understood as a limitation.

### EXAMPLE 1

### Combination of the progesterone receptor antagonist 11β-(4-acetylphenyl)-17β-hydroxy-17α-(1,1,2,2,2-pentafluoroethyl)-estra-4,9-dien-3-one together with Tamoxifen inhibits growth of breast cells with BRAC1 and BRCA2 knock down

MCF-10 mammary cells obtained from ATCC were treated with siRNA knocking down the BRCA1 and BRCA2 gene.

Cell growth in comparison to untransfected and mock transfected cells was compared. In a second step cells were stimulated either with progesterone and/or with estrogens. An increased proliferation was seen in the BRCA1 and BRCA1 ko cells in the presence of progesterone. The co-treatment with 11β-(4-acetylphenyl)-17β-hydroxy-17α-(1,1,2,2,2-pentafluoroethyl)-estra-4,9-dien-3-one alone or in combination with an antiestrogen was able to antagonize the effects of BRCA1 knock down. The effects on progesterone receptor protein expression were further investigated. By using siRNA for knock down of BRCA1 an increased stability of progesterone receptor which could be antagonized by the progesterone receptor antagonist 11β-(4-acetylphenyl)-17β-hydroxy-17α-(1,1,2,2,2-pentafluoroethyl)-estra-4,9-dien-3-one was found.

Thus, the results show that the combination of the progesterone-receptor antagonist 11β-(4-acetylphenyl)-17β-hydroxy-17α-(1,1,2,2,2-pentafluoroethyl)-estra-4,9-dien-3-one in combination with tamoxifen, according to the present invention results in a potent inhibition of the growth of BRCA1 knock down cells.

### EXAMPLE 2

### Combination of the Progesterone receptor antagonist 11β-(4-acetylphenyl)-17β-hydroxy-17α(1,1,2,2,2-pentafluoroethyl)-estra-4,9-dien-3-one together with the none-steroidal antiestrogen Tamoxifen

The combination of progesterone-receptor antagonist (11β-(4-acetylphenyl)-17β-hydroxy-17α(1,1,2,2,2-pentafluoroethyl)-estra-4,9-dien-3-one) with tamoxifen according to the present invention shows also synergistic effects in the treatment of chemically induced tumors (NMU-(Nitroso-methyl-urea) DMBA-(Dimethyl-benz-anthracene) model) in female rats.

Tumors were induced by a single intravenous injection of NMU (50 mg/kg) in female Sprague-Dawley rats (Tierzucht Schönwalde, age 50-55 days). Rats with at least one established tumor with a size of minimal 150 mm² were treated for 4 weeks by

1) Solvent control,
2) Ovariectomy (at treatment start),
3) The progesterone receptor antagonist 11β-(4-acetylphenyl)-17β-hydroxy-17α(1,1,2,2,2-pentafluoroethyl)-estra-4,9-dien-3-one, 1 mg/kg p.o.,
4) Tamoxifen, 1 mg/kg p.o.,
5) The progesterone receptor antagonist 11β-(4-acetylphenyl)-17β-hydroxy-17α(1,1,2,2,2-pentafluoroethyl)-estra-4,9-dien-3-one, 1 mg/kg p.o. and Tamoxifen, 1 mg/kg p.o. daily.

As parameter for growth inhibition the procentual change of tumor area determined by weekly caliper measurements was used. For statistical analysis of intergroup differences of mean values the Kruskal-Wallis-test was used.

Compared to the rapid growth in the control, ovariectomy resulted in an almost complete inhibition of tumor growth in the NMU-breast cancer model. Tumor growth inhibition by a standard Tamoxifen treatment (1 mg/kg p.o.) and single treatment with 11β-(4-acetylphenyl)-17β-hydroxy-17α(1,1,2,2,2-pentafluoro-ethyl)-estra-4,9-dien-3-one (1 mg/kg p.o.) was not significant different to the untreated control group. In contrast treatment with the combination of 11β-(4-acetylphenyl)-17β-hydroxy-17α(1,1,2,2,2-pentafluoroethyl)-estra-4,9-dien-3-one and Tamoxifen (both 1 mg/kg p.o.) resulted in a significant inhibition of tumor growth compared to the control.

The results are shown in Table I.

The results show that the combination has an synergistic effect in comparison to the single compounds.

TABLE I

| | Dose | |
|---|---|---|
| Compound | [mg/kg], p.o. | Median tumor weight [mg] *) |
| Control | — | 15200 |
| Ovariectomy | — | 0 |
| 11β-(4-acetylphenyl)- | 1 | 1800 |
| 17β-hydroxy- | | |
| 17α(1,1,2,2,2- | | |
| pentafluoroethyl)-estra- | | |
| 4,9-dien-3-one | | |
| Tamoxifen | 1 | 2950 |
| 11β-(4-acetylphenyl)- | 1 + 1 | 850 |
| 17β-hydroxy- | | |
| 17α(1,1,2,2,2- | | |
| pentafluoroethyl)-estra- | | |
| 4,9-dien-3-one and | | |
| Tamoxifen | | |

*) of 10 animals

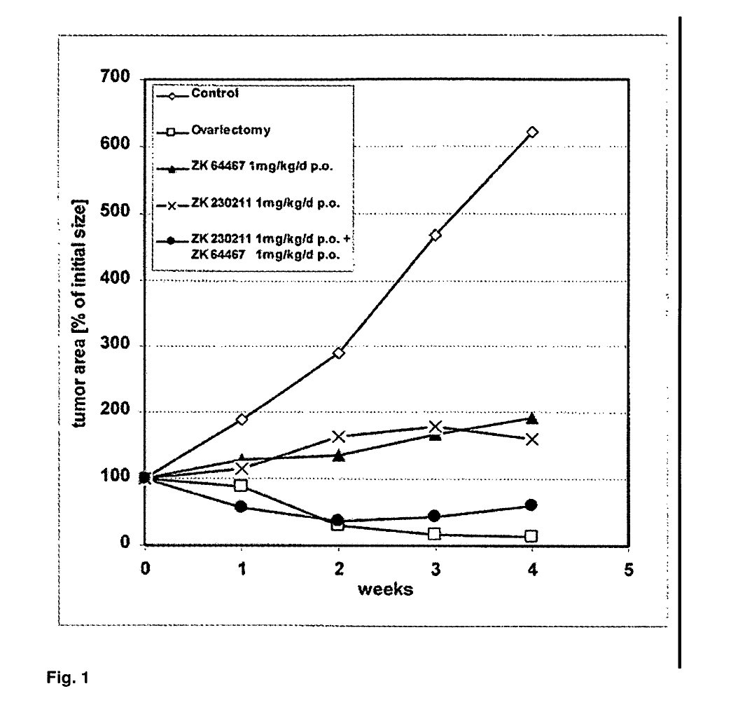

The effect of the combination comprising 11β-(4-acetylphenyl)-17β-hydroxy-17α(1,1,2,2,2-pentafluoroethyl)-estra-4,9-dien-3-one and Tamoxifen (1 mg/kg p.o.) in comparison to the single treatment on growth of NMU-induced rat mammary carcinomas and to ovariectomy is shown in FIG. 1/1.

ZK64467=Tamoxifen

ZK230211=11β-(4-acetylphenyl)-17β-hydroxy-17α(1,1,2,2,2-pentafluoro-ethyl)-estra-4,9-dien-3-one

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding European application No. 07090082.4, filed Apr. 23, 2007, and U.S. Provisional Application Ser. No. 60/914,385, filed Apr. 27, 2007, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A pharmaceutical combination comprising the progesterone-receptor antagonist 11β-(4-acetylphenyl)-17β-hydroxy-17α-(1,1,2,2,2-pentafluoroethyl)-estra-4,9-dien-3-one or a pharmaceutically acceptable derivative or analogue thereof together with at least one pure none-steroidal antiestrogen for the prophylaxis and treatment of BRCA1- or BRCA2-mediated breast cancer.

2. A pharmaceutical combination according to claim 1, wherein the pure antiestrogen is tamoxifen, raloxifene, droloxifen, toremifen, lasofoxifen, arzoxifen, GW5638, EM-800, idoxifen and basedoxifene.

3. A pharmaceutical combination according to claim 1, wherein the weight ratio of the progesterone-receptor antagonist and the none-steroidal antiestrogen is from 1:100 to 100:1.

4. A pharmaceutical combination according to claim 1, wherein the weight ratio of the progesterone-receptor antagonist and the none-steroidal antiestrogen or is from 1:4 to 4:1.

5. A pharmaceutical combination according to claim 1, wherein the progesterone-receptor antagonist is present in a unit dose of 0.1 to 100 mg and the none-steroidal antiestrogen or is present in a unit dose of 0.1 to 200 mg.

6. A pharmaceutical combination according to claim 1, wherein the progesterone-receptor antagonist is present in a unit dose of 10 to 150 mg and the none-steroidal antiestrogen or is present in a unit dose of 10 to 150 mg.

7. A pharmaceutically combination according to claim 1, wherein the progesterone-receptor antagonist and the none-steroidal antiestrogen is administered in the form of tablets, pills, dragees, gel capsules, granules, suppositories, implants, injectable sterile aqueous or oily solutions, suspensions, emulsions, ointments, creams, gels, patches for transdermal administration or formulations suitable for administration by inhalation.

8. A pharmaceutically combination according to claim 1, characterized that the combination comprises the progesterone-receptor antagonist 11β-(4-acetyl-phenyl)-17β-hydroxy-17α-(1,1,2,2,2-pentafluoroethyl)-estra-4,9-dien-3-one, a none-steroidal antiestrogen and a pharmacologically active agent.

9. A pharmaceutically combination according to claim 8, wherein the pharmacologically active agent is a cytotoxic agent.

10. A pharmaceutically combination according to claim 1 for oral administration.

11. A method for the prophylaxis or treatment of BRCA1- or BRCA2-mediated breast cancer, ovarian cancer endometrial cancer, gastric cancer, colorectal cancer, endometriosis, myeloma, myoma and meningioma comprising administering the combination according to claim 1.

12. A method of claim 11 for the treatment of BRCA1- or BRCA2-mediated breast cancer, ovarian cancer endometrial cancer, gastric cancer, colorectal cancer, endometriosis, myeloma, myoma and meningioma.
